(19) United States
(12) Patent Application Publication
(10) Pub. No.: US 2006/0163314 A1
(43) Pub. Date: Jul. 27, 2006

(54) Torsion adjusting module

(76) Inventor: Huan-Tsung Lin, Hsinchu (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/283,947

(22) Filed: Nov. 22, 2005

(30) Foreign Application Priority Data
Jan. 25, 2005 (TW) 94201375

(51) Int. Cl. A61B 17/04

(52) U.S. Cl. 227/179.1

(57) ABSTRACT

A torsion adjusting module is described. The module includes a shaft, a main body, an elastic member, and an adjusting device. The main body is made of an elastic material with an unclosed circular structure to couple to the shaft. A distance is formed at ends of the unclosed circular structure to keep a gap between the main body and the shaft. The elastic member is disposed in the gap between the shaft and the main body. The adjusting device is disposed at the ends of the unclosed circular structure to adjust the distance between the ends to adjust a torsion between the shaft and the main body. A predetermined torsion is set between the shaft and the main body before the adjusting device adjusts the adjusting device. The torsion is increased when the adjusting device is pressed to shrink the distance of the ends.

### RELATED APPLICATIONS

The present application is based on, and claims priority from, Taiwan Application Serial Number 94201375, filed on Jan. 25, 2005, the disclosure of which is hereby incorporated by reference herein in its entirety.

### BACKGROUND OF THE INVENTION

1. Field of the Invention

The invention relates to an adjusting module and, in particular, to a torsion adjusting module.

2. Description of the Related Art

To adjust torsion between two pivoting devices rotating with respect to each other and coupled by a shaft, a torsion adjusting module often provided between the shaft and the pivoting devices. The conventional torsion adjusting module usually is a fixing device penetrating through the pivoting devices. The fixing device, for example, has a set of nut and bolt or a rivet. The method of adjusting the torsion is to adjust the tightness between the nut and bolt, or the depth of the rivet. This kind of method for torsion adjusting is very hard to provide a stable torsion, because a contact area is narrow between the two pivoting devices and the head of bolt and the nut or between the two pivoting devices, the head of the rivet and the deformed rivet.

When rotating the pivoting devices and using the nut and the bolt as the fixing device, the nut and the bolt may loose or tie because of the friction between the fixing device and the devices. Therefore, it is hard to maintain a stable torsion.

When using the rivet as the fixing device, the rivet is hit to determine the depth of the rivet. Once a predetermined torsion is reached, it is hard to be loosened. The connection part is worn as the two pivoting devices rotate. Little by little, a gap is formed between them to weaken the torsion. A user has to hit the rivet again to adjust the torsion. If the user wants to disassemble the two pivoting devices, the rivet has to be destroyed. The destroyed rivet can no longer be used in the torsion adjusting module. That is, a new rivet has to be used in order to replace the destroyed rivet.

If the two pivoting devices have respective holes with different size from the hole of the fixing device, the two pivoting devices cannot rotate about the same center. It is very difficult to adjust orientations of the pivoting devices with high precision.

### SUMMARY OF THE INVENTION

Therefore, an objective of the invention is to provide a torsion adjusting module that uses a coupling surface between the shaft and a main body of the torsion adjusting module to control the torsion.

Another objective of the invention is to provide a torsion adjusting module assembled from components that are easy to disassemble.

A further objective of the invention is to provide an adjusting base that uses a torsion adjusting module with good coaxiality to adjust the orientation of a supported apparatus with precision.

To achieve the above objectives, the module of the present invention includes a shaft, a main body, an elastic member, and an adjusting device. The main body is made of an elastic material with an unclosed circular structure having a circular through hole for coupling to the shaft. A distance is formed between two ends of the unclosed circular structure to keep a gap between the main body and the shaft. The elastic member is disposed between the shaft and the main body. The adjusting device is disposed at the ends of the unclosed circular structure to adjust the distance between the ends so as to adjust a torsion between the shaft and the main body.

A predetermined torsion provided from the elastic member is set between the shaft and the main body before the adjusting device adjusts the main body. When the adjusting device is rotated so as to shrink the distance between the ends of the unclosed circular structure, the gap between the main body and the shaft is shortened. The elastic member is compressed by the main body, and then the elastic member presses the shaft to increase the torsion between the main body and the shaft.

### BRIEF DESCRIPTION OF THE DRAWINGS

These and other features, aspects and advantages of the invention become apparent by reference to the following description and accompanying drawings which are given by way of illustration only, and thus are not limitative of the invention, and wherein:

FIG. 1A is a top view of the disclosed torsion adjusting module of the present invention;

FIG. 1B is a cross-sectional view of A-A in FIG. 1A;

FIG. 2 is a perspective view of the disclosed torsion adjusting module of the present invention;

FIG. 3 is an exploded view of the disclosed torsion adjusting module of the present invention;

FIG. 4A is a schematic view of a main body with one opening pressing on the shaft of the present invention;

FIG. 4B is a schematic view of a main body with two openings pressing on the shaft of the present invention;

FIG. 4C is a schematic view of a main body with three openings pressing on the shaft of the present invention;

FIG. 4D is a top view of the disclosed torsion adjusting module with the openings formed on an outer side surface of the main body of the present invention;

FIG. 5 is a top view of the disclosed torsion adjusting module with pads of the present invention; and

FIG. 6 shows one embodiment of the adjusting base having three of the disclosed torsion adjusting modules of the present invention.

### DETAILED DESCRIPTION OF THE PREFERRED EMBODIMENTS

The present invention is apparent from the following detailed description, which proceeds with reference to the accompanying drawings, the same references relate to the same elements.

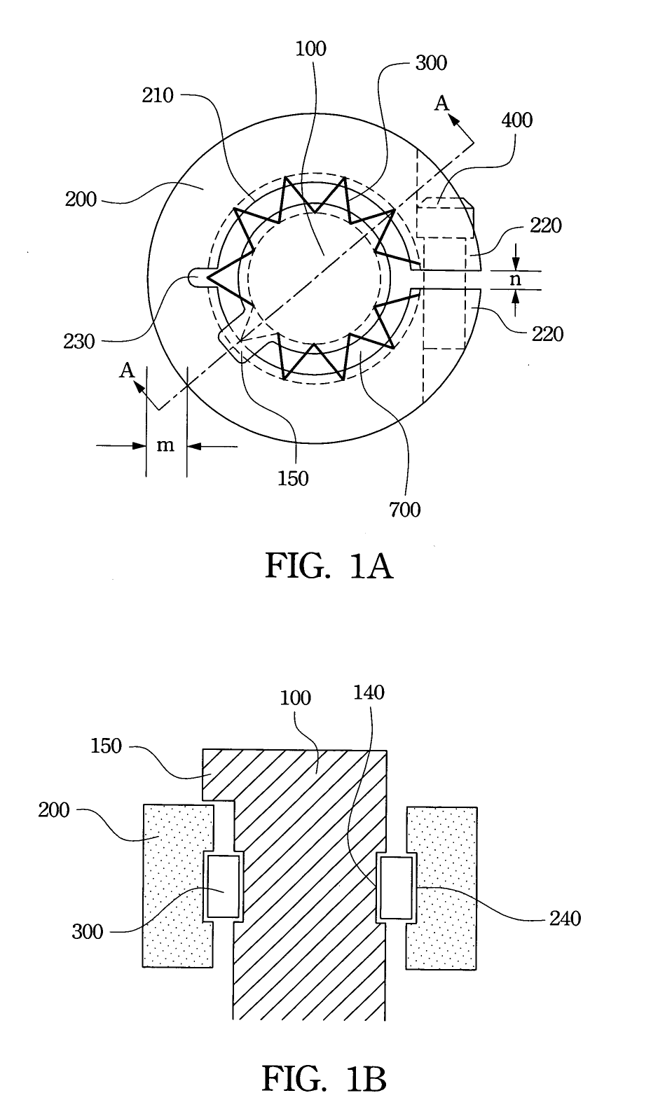

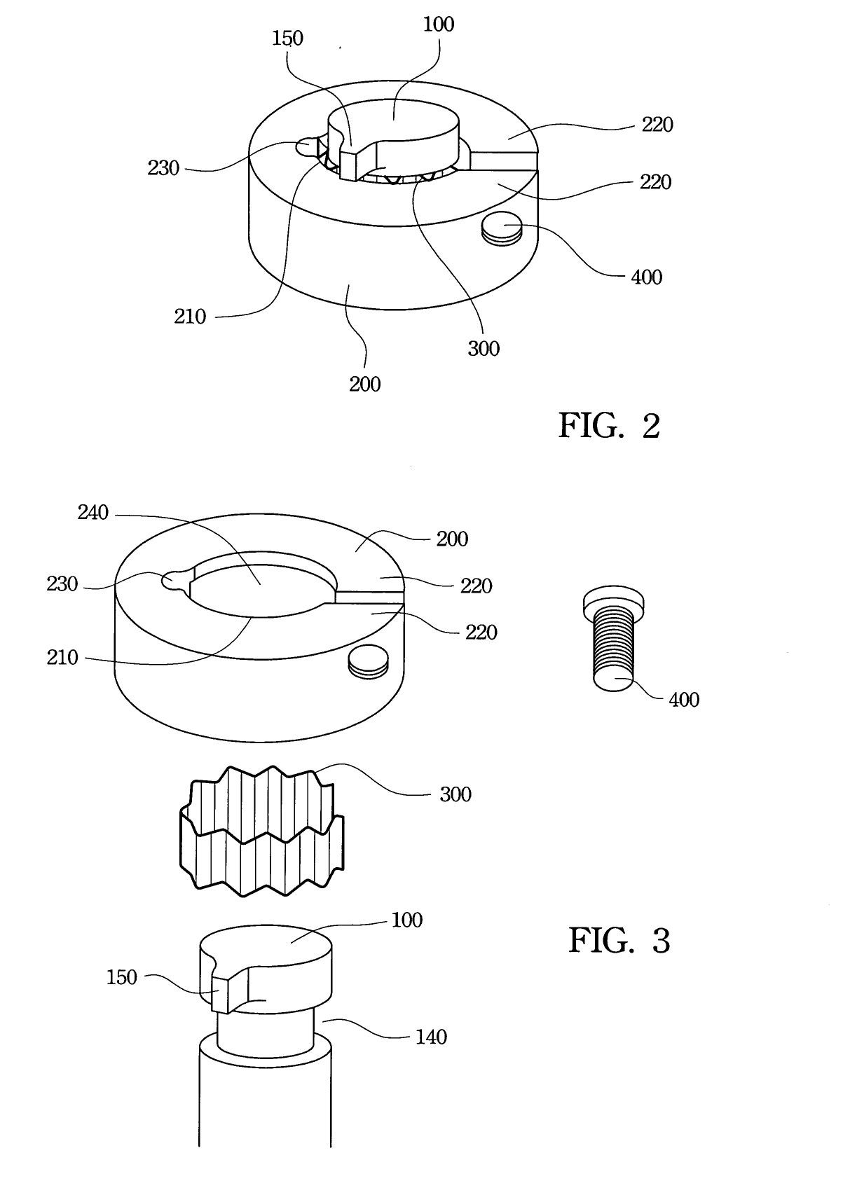

As shown in FIGS. 1A, 1B, 2, and 3, a disclosed torsion adjusting module contains a shaft 100, a main body 200, an elastic member 300, and an adjusting device 400.

The main body 200 has an unclosed circular structure to couple to the shaft 100, with a circular through hole 210 formed at a center of the main body 200. A diameter of the through hole 210 is slightly larger than a diameter of the shaft 100 to couple to the shaft 100. The main body 200 is made of an elastic material. A distance n is formed between two ends 220 of the unclosed circular structure to keep a gap 700 between the main body 200 and the shaft 100.

The elastic member 300 is disposed in the gap 700 between the shaft 100 and the main body 200. The elastic member 300 is a wavy elastic member in contact with the shaft 100 and the main body 200 by the troughs and crests of the elastic member 300. A variation of the elastic member 300 is homogeneously distributed in the gap 700 and making the centers of the shaft 100 and the through hole 210 of the main body 200 coincide to maintain good coaxiality and increase a stability of a torsion between the shaft 100 and the main body 200. The elastic member 300 is made of a highly elastic material. The hardness of the highly elastic material for the elastic member 300 is smaller than the hardness of the shaft 100 and the main body 200 to reduce a wear of the shaft 100 and the main body 200 during the shaft 100 and the main body 200 rotate. The highly elastic material can be heat treated phosphor bronze to prevent from an elastic restoration of the wavy structure.

The adjusting device 400 is disposed at two ends 220 of the unclosed circular structure to adjust the distance n between two ends 220 so as to adjust the torsion between the shaft 100 and the main body 200. The adjusting device 400 is an adjusting device with threads.

A predetermined torsion is set by the elastic member 300 between the shaft 100 and the main body 200 before the adjusting device 400 adjusts the main body 200. The torsion is increased when the elastic member 300 is compressed to press the shaft 100, as a result of a shrinking diameter of the through hole 210 to shorten the distance n between two ends 220 of the unclosed circular structure.

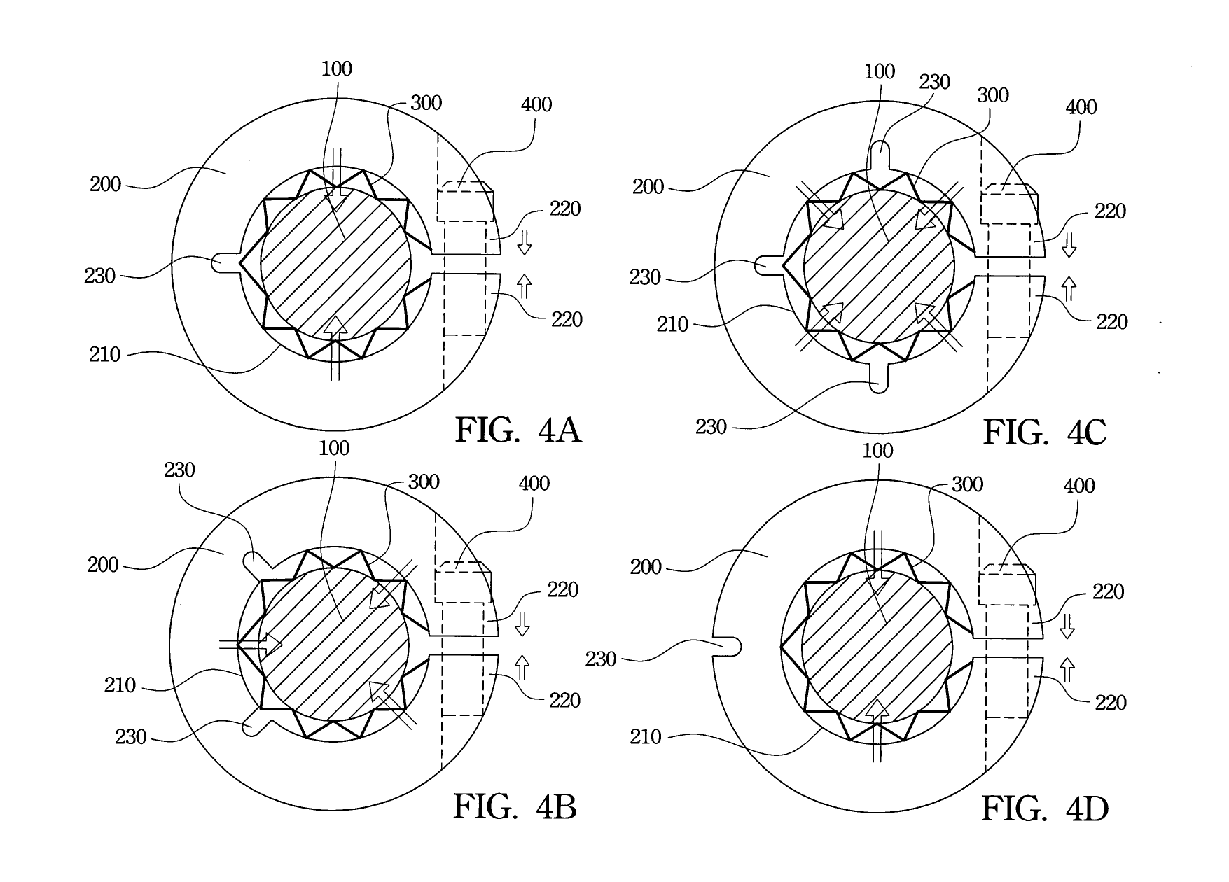

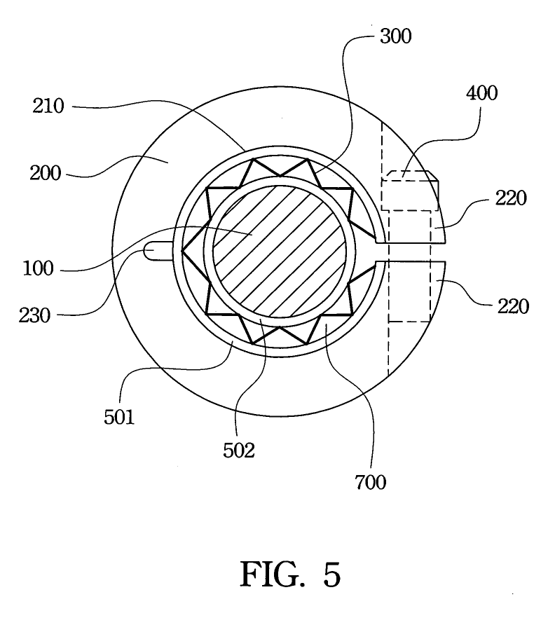

The main body 200 further contains at least one opening 230 formed on the unclosed circular structure at equal intervals. A distance between a bottom of an opening 230 and a wall of the through hole 210 is smaller than a thickness m of the wall of the unclosed circular structure. When the adjusting device 400 shortens the distance n between the ends 220, the variation of the main body 200 is distributed at the openings 230, so that the main body 200 presses homogeneously directly on the elastic member 300 and indirectly on the shaft 100. The more opening 230 there is, the more homogeneous pressure on the elastic member 300 and the shaft 100 is, as shown in FIGS. 4A to 4C. As shown in FIG. 4D, the openings 230 can be formed on an outer side surface of the main body 200.

As shown in FIG. 1B and 3, the shaft 100 has an accommodation space 140 formed on a circumference of a part wall of shaft where the shaft 100 couples to the through hole 210 to accommodate the elastic member 300 and prevent the elastic member 300 from departing from the shaft 100. Alternatively, the main body 200 has an accommodation space 240 formed on the circumference of the wall of the through holes 210 to accommodate the elastic member 300 and prevent the elastic member 300 from departing from the main body 200. Furthermore both the shaft 100 and the main body 200 contain the accommodation spaces 140, 240, formed respectively on the walls where the shaft 100 and the main body 200 are coupled. Such a configuration can prevent the elastic member 300 from departing form the gap 700 between the shaft 100 and the main body 200. Moreover, the contact between the elastic member 300 and the accommodation spaces 140, 240 can prevent the shaft 100 and the main body 200 from axial deviations.

The shaft 100 also contains a blocking portion 150 installed on one end of the shaft 100. The blocking portion 150 protrudes from the wall of the shaft 100 and without penetrating through the through hole 210 and the openings 230 to prevent the main body 200 from sliding out of the shaft 100 from the end disposed the blocking portion 150.

As shown in FIG. 5, pads 501, 502 made of a wear-resistant material are further disposed between the main body 200 and the elastic member 300 and/or between the elastic member 300 and the shaft 100 to reduce the frictional wear among the main body 200, the elastic member 300, and the shaft 100 during rotations.

Within the scope of the invention, the shapes of various components are not limited to those shown in the drawings. As long as the disclosed functions can be achieved, the components can have any desired shapes.

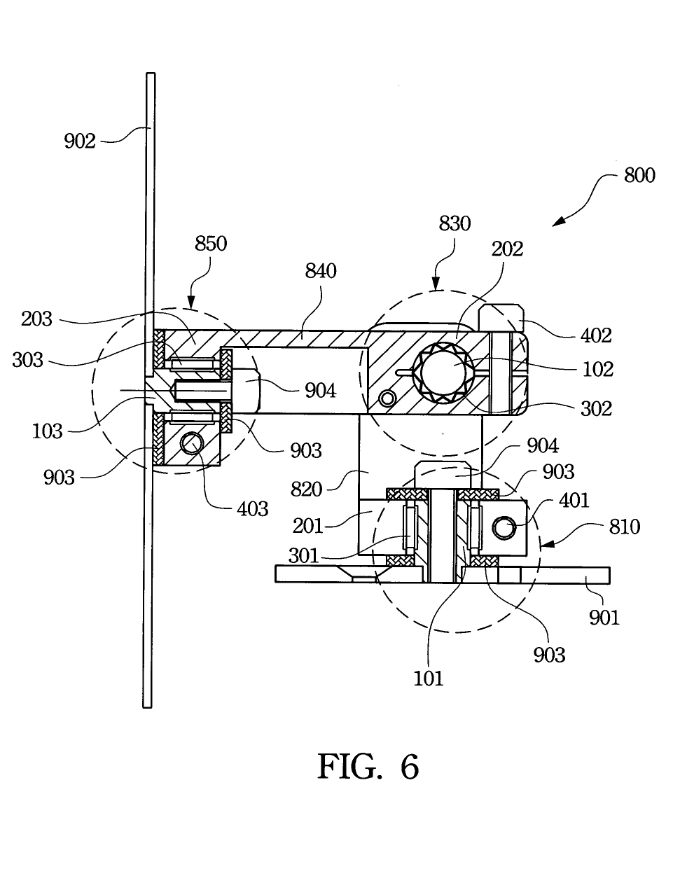

The invention can be applied to various apparatuses. For example, the invention is used in an adjusting base, as shown in FIG. 6. The adjusting base includes a base 901 and a movable arm 800. The movable arm 800 contains a first torsion adjusting module 810, a first arm 820, a second torsion adjusting module 830, a second arm 840, and a third torsion adjusting module 850.

The shaft 101 of the first torsion adjusting module 810 is perpendicularly coupled to the base 901. One end of the first arm 820 is coupled to the main body 201 of the first torsion adjusting module 810. An extension line of an axis of a shaft 102 of the second torsion adjusting module 830 is perpendicularly to an extension line of an axis of the shaft 101 of the first torsion adjusting module 810. They meet and couple at the other end of the first arm 820. One end of the second arm 840 is coupled to the main body 202 of the second torsion adjusting module 830. The main body 203 of the third torsion adjusting module 850 is coupled to the other end of the second arm 840. An extension line of an axis of a shaft 103 of the third torsion adjusting module 850 and the extension line of the axis of the shaft 102 of the second torsion adjusting module 830 are coupled perpendicularly. A support apparatus 902, such as a thin display device, is coupled perpendicularly to the shaft 103 of the third torsion adjusting module 850. Pads 903 are provided among the torsion adjusting modules 810, 830, 850, the arms 820, 840, the base 901, and the apparatus 902. The end surfaces of the shafts 101, 102, 103 are fixed by a fixing device 904 and the pads 903, so that each torsion adjusting module has smooth motions and all the elements are prevented from departure. The pads 903 and the fixing device 904 of the second torsion adjusting module are provided on the other end of the shaft 102 and thus are not shown in the figure. Thus, the first torsion adjusting module 810 is used to adjust a rotating angle of the apparatus 902 in the left and right directions. The second torsion adjusting module 830 is used to adjust the rotating angle of the apparatus 902 in the up and down directions. The third torsion adjusting module 850 is used to adjust the horizontal angle of the apparatus 902. The arms are used to extend the distances between the torsion adjusting modules or between the apparatus and the torsion adjusting modules.

In FIG. 6, the first torsion adjusting module 810 has the shaft 101 coupled with the base 901, and the main body 201 coupled to the first arm 820. However, the first adjusting module 810 can have the main body 201 coupled to the base 901, and the shaft 101 coupled to the first arm 820. The coupling means between the second torsion adjusting module 830 and the third torsion adjusting module 850 can also be achieved by exchanging the coupling positions of the shafts 102, 103 and the main bodies 202, 203.

In the preferred embodiment, there are three torsion adjusting modules to constitute a multi-joint movable arm. In accord with practical situations, the number of torsion adjusting modules and multi-joint movable arms can be increased or decreased. For example, if there is only one torsion adjusting module, the arm can be eliminated and the apparatus is directly coupled to the torsion adjusting module. Alternatively, other types of torsion adjusting modules are be used. Such variations should be construed as part of the invention.

From the preferred embodiment, the invention has the following advantages:
1. The coupling surface between the shaft and the torsion adjusting module controls the torsion to increase the stability of the torsion.
2. Simple collapsible components are assembled to form the torsion adjusting device for the convenience of element replacement and maintenance.
3. The adjusting base formed from the torsion adjusting module with good coaxiality can be used to adjust the orientation of a suppoted apparatus with high precision.

While the present invention has been described by way of example and in terms of the embodiment, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A torsion adjusting module, comprising:
a shaft;
a main body, having an unclosed circular structure with a circular through hole formed at a center of the unclosed circular structure for coupling to the shaft, two ends of the unclosed circular structure being kept at a distance to form a gap between the shaft and the main body;
an elastic member disposed in the gap between the shaft and the main body; and
an adjusting device disposed at the ends of the unclosed circular structure to adjust a distance between the ends and the torsion between the shaft and the main body;
wherein a predetermined torsion is set between the shaft and the main body before the adjusting device adjusts the main body, and the torsion is increased when the adjusting device adjusts the distance between the ends to shorten the through hole compressing the elastic member to press the shaft.

2. The torsion adjusting module of claim 1, wherein the elastic member is a wavy elastic member.

3. The torsion adjusting module of claim 1, wherein the elastic member is made of an elastic material and a hardness of the elastic material is smaller than hardness of the shaft and the main body.

4. The torsion adjusting module of claim 1, wherein the elastic member is made of phosphor bronze.

5. The torsion adjusting module of claim 1, wherein the main body comprises at least one opening formed at equal intervals on the unclosed circular structure, a distance between a bottom of the openings and a wall of the through hole is smaller than the thickness of the wall of the unclosed circular structure, when the adjusting device shrinks the distance between the ends of unclosed circular structure, a variation of the main body is distributed to the opening for the main body to homogeneously press directly on the elastic member and indirectly on the shaft.

6. The torsion adjusting module of claim 1, wherein the shaft comprises an accommodation space formed on a circumference of a part wall of the shaft to accommodate the elastic member to prevent the elastic member from departing from the shaft.

7. The torsion adjusting module of claim 1, wherein the main body comprises an accommodation space formed on a circumference of the through hole to accommodate the elastic member to prevent the elastic member from departing from the main body.

8. The torsion adjusting module of claim 1, wherein the shaft and the main body respectively have an accommodation space, formed respectively on the walls where the shaft and the main body are coupled, to prevent the elastic member from departing from the gap between the shaft and the main body, and the contact between the elastic member and the accommodation spaces prevents the shaft and the main body from axial deviations.

9. The torsion adjusting module of claim 1, wherein the shaft comprises a blocking portion disposed on one end of the shaft and protruding from the wall of the shaft to prevent the main body from sliding out of the shaft from the end disposed the blocking portion.

10. The torsion adjusting module of claim 1, wherein at least one pad made of a wear-resistant material are further provided respectively between the main body and the elastic member and between the elastic member and the shaft to reduce the wear among the main body, the elastic member, and the shaft during rotations of the main body, the elastic member, and the shaft.

11. An adjusting base for supporting an apparatus and adjusting an orientation of the apparatus, comprising:
a base; and
a torsion adjusting module disposed between the base and the apparatus, comprising:
a shaft;
a main body, having an unclosed circular structure with a circular through hole formed at a center of the unclosed circular structure for coupling to the shaft, two ends of the unclosed circular structure being kept at a distance to form a gap between the shaft and the main body;
an elastic member disposed in the gap between the shaft and the main body; and
an adjusting device disposed at the ends of the unclosed circular structure to adjust the distance between the ends to adjust the torsion between the shaft and the main body.

12. The adjusting base of claim 11, further comprising an arm disposed between the apparatus and the torsion adjusting module to extend a distance between the apparatus and the torsion adjusting module.

13. The adjusting base of claim 11, wherein the apparatus is perpendicularly coupled to the shaft.

14. The adjusting base of claim 11, wherein the elastic member is a wavy elastic member.

15. The adjusting base of claim 11, wherein the main body further comprises at least one opening formed at equal intervals on the unclosed circular structure, a distance between a bottom of the opening and a wall of the through hole is smaller than a thickness of a wall of the unclosed circular structure, when the adjusting device shrinks the distance between the ends of unclosed circular structure, a variation of the main body is distributed to the opening for the main body to homogeneously press directly on the elastic member and indirectly on the shaft.

16. The adjusting base of claim 11, wherein the apparatus is a thin display device.

17. An adjusting base, comprising:
abase; and
a movable arm provided between the base and an apparatus, the movable arm comprising:
a plurality of torsion adjusting modules, each torsion adjusting module comprising:
a shaft;
a main body, having an unclosed circular structure with a circular through hole formed at a center of the unclosed circular structure for coupling to the shaft, two ends of the unclosed circular structure being kept at a distance to form a gap between the shaft and the main body;
an elastic member disposed in the gap between the shaft and the main body; and
an adjusting device disposed at the ends of the unclosed circular structure to adjust the distance between the ends, to adjust the torsion between the shaft and the main body; and
a plurality of arms coupled respectively to the torsion adjusting modules to form multijoint movable arms for supporting the apparatus and adjusting the orientation of the apparatus.

18. The adjusting base of claim 17, wherein the elastic member is a wavy elastic member.

19. The adjusting base of claim 17, wherein the main body further comprises at least one opening formed at equal intervals on the unclosed circular structure, a distance between a bottom of the opening and a wall of the through hole is smaller than a thickness of a wall of the unclosed circular structure, when the adjusting device shrinks the distance between the ends of the unclosed circular structure, a variation of the main body is distributed to the opening for the main body to homogeneously press directly on the elastic member and indirectly on the shaft.

20. The adjusting base of claim 17, wherein the apparatus is a thin display device.
